(19) United States
(12) Patent Application Publication
(10) Pub. No.: US 2022/0401242 A1
(43) Pub. Date: Dec. 22, 2022

(54) MEDICAL DEVICE LOADING TOOL

(71) Applicant: BOSTON SCIENTIFIC SCIMED, INC., Maple Grove, MN (US)

(72) Inventors: Tim O'Connor, Galway (IE); Declan Loughnane, Galway (IE); John Lardner, Eircode (IE); Jack Walsh, Galway (IE); Cornelia Galwey, Galway (IE); Enda Hannon, Galway (IE); Pearse A. Coffey, Galway (IE)

(73) Assignee: BOSTON SCIENTIFIC SCIMED, INC., Maple Grove, MN (US)

(21) Appl. No.: 17/840,838

(22) Filed: Jun. 15, 2022

Related U.S. Application Data
(60) Provisional application No. 63/211,328, filed on Jun. 16, 2021.

(51) Int. Cl. A61F 2/95; A61F 2/24

(52) U.S. Cl. CPC A61F 2/9524; A61F 2/2418; A61F 2/2476

(57) ABSTRACT

Example medical device loading devices are disclosed. An example loading device for a stent-valve includes an elongated body having a proximal end region, a distal end region and a lumen extending therein. The loading device also includes a collar coupled to the distal end region of the body, the collar including first end region, a second end region and a lumen extending therein. Further, the loading device includes a first compression assembly coupled to the collar, wherein the first compression assembly is configured to shift between a first position a second position. The loading device also includes a second compression assembly coupled to the collar, wherein the second compression assembly is configured to shift between a third position and a fourth position.

### CROSS REFERENCE TO RELATED APPLICATIONS

This application claims the benefit of priority of U.S. Provisional Application No. 63/211,328 filed Jun. 16, 2021, the entire disclosure of which is hereby incorporated by reference.

### TECHNICAL FIELD

The present disclosure pertains to a loading tool for loading a medical device into a delivery device. In some examples, the medical device may include a stent-valve, however, the loading tool may also be utilized to load other types of medical devices into delivery devices.

### BACKGROUND

The present disclosure describes a loading tool for loading a stent-valve into a medical device delivery system. The loading tool may compress the stent-valve into a configuration which is suitable to be accommodated within two complementary sheaths located at the distal end region of a delivery catheter. Additionally, the two sheaths may cover different portions of the stent-valve and may be translatable in opposite directions (relative to one another) to deploy a distal end region and a proximal end region of the stent-valve in a predetermined sequence. Upon deployment, the stent-valve may expand to a deployed state.

It can be appreciated that loading the stent-valve on to the delivery catheter may be an intricate process. For example, damage may result during the loading process from over compression, nonuniform stress distribution, buckling, non-circularity during compression, and/or from tearing or abrasion of valve component tissue. Further, a deformed or damaged stent-valve may function imperfectly, or have a reduced operational life, or may be difficult or even impossible to implant correctly. The complications may be exacerbated in the case of a self-expanding type of stent-valve as a self-expanding stent-valve may have a strong restoration force when compressed, and therefore, requires application of a large compression force to compress the stent-valve down to its compressed condition. Additionally, complications may arise when the stent-valve is to be compressed for loading on to a delivery catheter having multiple sheaths that close over different portions of the stent-valve. Therefore, it may be beneficial to utilize a multi-clamp stent-valve loading tool which not only simplifies the loading process, but also reduces the likelihood of damage to the stent-valve during the loading process. Example stent-valve loading tools which utilize multi-clamp fixtures are disclosed herein.

### BRIEF SUMMARY

This disclosure provides design, material, manufacturing method, and use alternatives for medical devices. An example loading device for a stent-valve includes an elongated body having a proximal end region, a distal end region and a lumen extending therein. The loading device also includes a collar coupled to the distal end region of the body, the collar including first end region, a second end region and a lumen extending therein. Further, the loading device includes a first compression assembly coupled to the collar, wherein the first compression assembly is configured to shift between a first position a second position. The loading device also includes a second compression assembly coupled to the collar, wherein the second compression assembly is configured to shift between a third position and a fourth position.

Alternatively or additionally to any of the embodiments above, wherein the first compression assembly is longitudinally offset from the second compression assembly.

Alternatively or additionally to any of the embodiments above, wherein the first compression assembly and the second compression assembly are configured to maintain the stent-valve in a radially compressed state when in the first position.

Alternatively or additionally to any of the embodiments above, wherein shifting first compression assembly, the second compression assembly, or both the first compression assembly and the second compression assembly from the first position to the second position includes releasing the stent-valve from the radially compressed state.

Alternatively or additionally to any of the embodiments above, wherein the first compression assembly is configured to release the stent-valve from the radially compressed state while the second compression assembly maintains the stent-valve in radially compressed state.

Alternatively or additionally to any of the embodiments above, wherein the second compression assembly is configured to release the stent-valve from the radially compressed state after the first compression assembly releases the stent-valve from the radially compressed state.

Alternatively or additionally to any of the embodiments above, wherein the first compression assembly includes a first compression member coupled to a first actuation member.

Alternatively or additionally to any of the embodiments above, wherein actuation of the first actuation member shifts the first compression member between the first position and the second position.

Alternatively or additionally to any of the embodiments above, further comprising a second compression member coupled to a second actuation member, and wherein both the first actuation member and the second actuation member include a threaded pin.

Alternatively or additionally to any of the embodiments above, wherein the first actuation member includes a cam.

Alternatively or additionally to any of the embodiments above, wherein the first actuation member includes compression lever.

Alternatively or additionally to any of the embodiments above, wherein the compression lever includes a first projection and a second projection, and wherein the first projection is configured to releasably engage with the second projection.

Alternatively or additionally to any of the embodiments above, wherein the first compression member includes a compression ring, and wherein the circumference of the compression ring is circumferentially discontinuous.

Alternatively or additionally to any of the embodiments above, wherein the compression ring includes an aperture having a first diameter when the first compression assembly is in the first position, and wherein the aperture has a second diameter when the first compression assembly is in the second position, and wherein the first diameter is less than the second diameter.

Another example loading device for a stent-valve includes an elongated body having a proximal end region, a distal end region and a conical lumen extending therein. The loading device also includes a collar coupled to the distal end region of the body, the collar including first end region, a second end region and a lumen extending therein. The loading device also includes a first compression assembly coupled to the collar, a second compression assembly coupled to the collar, wherein the first compression assembly is longitudinally offset from the second compression assembly. Further, the first compression assembly and the second compression assembly are configured to maintain the stent-valve in a radially compressed state while in a first position. Additionally, the first compression assembly is configured to release the stent-valve from the radially compressed state while the second compression assembly maintains the stent-valve in the radially compressed state.

Alternatively or additionally to any of the embodiments above, wherein the first compression assembly includes a first compression member coupled to a first actuation member.

Alternatively or additionally to any of the embodiments above, wherein the first actuation member includes a cam.

Alternatively or additionally to any of the embodiments above, wherein the first actuation member includes compression lever.

Alternatively or additionally to any of the embodiments above, wherein the first compression member includes a compression ring, and wherein the circumference of the compression ring is circumferentially discontinuous.

An example method of loading a stent-valve on to a stent-valve delivery device includes positioning a first sheath of the stent-valve delivery device adjacent to a stent-valve loading device, the stent-valve loading device including an elongated body and a collar coupled to the distal end region of the body. The stent device also includes a first compression assembly coupled to the collar, the first compression assembly including a first compression region. Further, the stent device also includes a second compression assembly coupled to the collar, the second compression assembly including a second compression region. Additionally, the method further includes compressing the stent-valve within the first compression region and the second compression region, releasing the stent from the first compression region, advancing the first sheath of the stent-valve delivery device over a first portion of the stent-valve, releasing the stent from the second compression region and advancing the first sheath of the stent-valve delivery device over a second portion of the stent-valve.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates an example stent-valve delivery system including an example stent-valve;

FIG. 2 illustrates the stent-valve and the stent-valve delivery system of FIG. 1 in a loaded configuration;

FIG. 3 illustrates a portion of the stent-valve delivery system of FIG. 2;

FIG. 4 illustrates an example medical device loading tool;

FIG. 5 is a partial cross-sectional view of the example loading tool shown in FIG. 4;

FIG. 6 illustrates a front view of the loading tool shown in FIG. 4 in a first position;

FIG. 7 illustrates a front view of the loading tool shown in FIG. 4 in a second position;

FIGS. 8-11 illustrate an example methodology of loading an example stent-valve onto an example stent-valve delivery system using the loading tool illustrated in FIG. 4;

FIG. 12 illustrates another example loading tool;

FIG. 13 is a partial cross-sectional view of the example loading tool shown in FIG. 12;

FIG. 14 illustrates a front view of the loading tool shown in FIG. 13 in a first position;

FIG. 15 illustrates a front view of the loading tool shown in FIG. 13 in a second position;

FIG. 16 illustrates another example stent-valve loading tool in a first position;

FIG. 17 illustrates the example stent-valve loading tool shown in FIG. 16 in a second position;

FIG. 18 illustrates another example stent-valve loading tool;

FIG. 19 illustrates another example stent-valve loading tool in a first position;

FIG. 20 illustrates the example stent-valve loading tool shown in FIG. 18 in a second position;

FIG. 21 illustrates another stent-valve loading tool.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

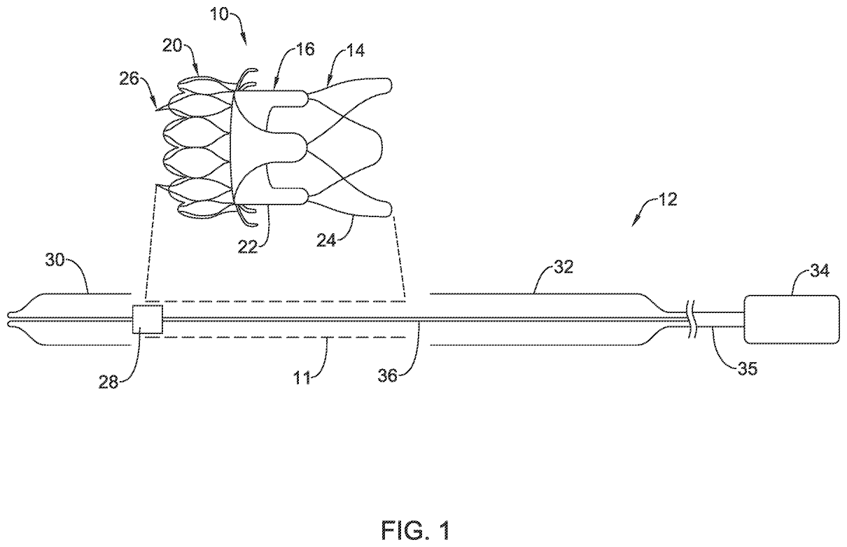

FIG. 1 illustrates an example stent-valve 10. The stent-valve 10 may be a cardiac stent-valve (e.g., an aortic stent-valve). The stent-valve 10 may be configured for transcatheter implantation in the body. For example, the stent-valve 10 may be configured to implantation in the body via the use of minimally invasive techniques. Further, the stent-valve 10 may be configured for transcatheter aortic valve replacement (“TAVR”). Although a particular geometry of stent-valve 10 is disclosed herein, it will be appreciated that the invention is not limited to any specific stent-valve geometry.

It can be appreciated that the stent-valve 10 may be transformable between an expanded configuration (as illustrated in FIG. 1), and a compressed configuration. The compressed configuration of the stent-valve 10 is depicted by the dashed line 11 in FIG. 1. The expanded configuration of the stent-valve 10 may correspond approximately to a deployed (e.g., operative) configuration of the stent-valve 10 after deployment and implantation. It can be appreciated that the compressed configuration 11 may correspond to a delivery state to be accommodated by a delivery catheter 12 and/or for introduction into the anatomy to the desired implantation site.

Additionally, the stent-valve 10 may be self-expanding, and therefore, resiliently biased towards the expanded configuration. Further, the stent-valve may be compressible to the compressed configuration 11 via application of radial compression forces. The stent-valve 10 may remain in its compressed state while constrained within a portion of the stent-valve delivery catheter 12. When released (e.g., deployed) from the constraints of the delivery catheter 12, the stent-valve 10 may expand radially outward to an expanded (e.g., deployed) configuration. Alternatively, it can be appreciated that the stent-valve 10 may be of a non-self-expanding stent-valve that requires application of an expansion force to shift the stent-valve 10 from the compressed configuration 11 to the expanded (e.g., deployed) configuration.

FIG. 1 further illustrates that the stent-valve 10 may include a stent component 14 and a valve component 16. The stent component 14 may provide an anchoring function for anchoring the stent-valve 10 in the body and/or a support function for supporting the valve component 16. The stent component 14 may be of any suitable material or materials. For example, the stent component 14 may be constructed from a metal. However, this is not intended to be limiting. Rather, it can be appreciated that the stent component 14 may be constructed from a polymeric material or combinations of metallic and polymeric materials. Example materials may include shape memory and/or superelastic alloys (for example, nitinol), stainless steel, or cobalt-chromium alloy.

Additionally, the stent component 14 may include any geometry desired for anchoring and/or aligning the stent-valve 10 with respect to the anatomy at a desired implantation site. In some examples, the stent component 14 may be generally cylindrical in shape, or include one more generally cylindrical portions or portions lying on a generally cylindrical surface. Additionally, the stent component 14 may be generally non-cylindrical in shape or include one or more generally non-cylindrical portions or portions lying on a non-cylindrical surface. Further, the stent component 14 may comprise one or more anchor projections, stabilization portions, or strengthening members.

As shown in FIG. 1, the stent component 14 may also include a valve support portion 22. The valve support portion 22 may include a framework (e.g., scaffold) designed to support the valve component 16. For example, it can be appreciated that the stent component 14 may generally include a shape that follows a peripheral contour of the valve component 16.

In some examples, the stent component 14 may further include a stabilization or alignment portion 24 defined, for example, by a plurality of wings or arches 24. For example, FIG. 1 illustrates that the stent component 14 may include three wings or arches 24. The arches 24 may extend from the tips of the valve support portion 22, to define a vaulted structure thereover. The alignment portion 24 may have a greater flexibility than the anchoring portion 20 and/or the valve support function 22. Further, the alignment portion 24 may have a resistance to compression that is smaller than the resistance to compression of the anchoring portion 20. Additionally, the alignment portion 24 may be more flexible (e.g., less rigid) than the anchoring portion 20 and/or the valve support portion 22. For example, the alignment portion 24 may be more flexible when compressed radially than the anchoring portion 20 and/or the valve support portion 22.

As will be discussed in greater detail below, the stent component 14 may also include one or more attachment portions 26 for attaching the stent component 14 to a stent receiver 28 of the delivery catheter 12. In some examples, the stent receiver 28 may be a stent holder and may be referred to as such hereinafter, although other types of receivers for receiving and/or accommodating at least a portion of the stent-valve 10 may be used as desired. Additionally, the attachment portions 26 may include one or more geometrical openings, or one or more lugs or other projections, for forming an interference (e.g. interlocking) fit with a complementary portion of the stent holder 28. The attachment portions 26 may be arranged at or adjacent to a distal end region of the stent component 14. In the example shown in FIG. 1, the attachment portions 26 may defined by one or more extensions of cells of the anchoring portion 20. In other words, the attachment portions 26 may be formed from the wires, struts, filaments, etc. used to form the anchoring portion 20.

Additionally, the valve component 16 may be of any suitable natural and/or synthetic material. For example, the valve component 16 may include porcine and/or bovine pericardium and/or harvested natural valve material. The valve component 16 may further include a plurality of leaflets arranged to collapse (e.g., fold, engage, etc.) into a closed position to obstruct flow in one direction, while flexing apart to an open position to allow flow in an opposite direction. As discussed above, the valve component 16 may be generally coupled to the valve support portion 22 and/or at least be positioned within at least a portion of the anchoring portion 20. In some examples, the stent-valve 10 (including the valve component 16) may further include an inner skirt and/or an outer skirt covering at least partly a respective inner or outer surface portion of the stent component 14. For example, the inner skirt and/or an outer skirt may cover at least a portion of the anchoring portion 20 and/or at least a portion of the valve support portion 22.

FIG. 1 further illustrates that the distal end region of the delivery catheter 12 may include multiple sheaths which together define a containment region of the delivery catheter 12. It can be appreciated that the multiple sheath of the containment region may be designed to accommodate the stent-valve 10. FIG. 1 illustrates that, in some examples, the delivery catheter 12 may include a first (e.g. distal) sheath 30 and a second (e.g. proximal) sheath 32. Together, the first sheath 30 and the second sheath 32 may be configured to cover respective portions of the stent-valve 10 in its compressed state 11, thereby restricting the stent-valve 10 from radial expansion until the first sheath 30 and/or the second sheath 32 are manipulated to release the respective portions of the stent-valve 10 for which they are covering. For example, the first sheath 30 may cover the attachment portion 26 and/or a portion of the anchoring portion 20, and the second sheath 32 may cover a portion of the anchoring portion 20, the valve support portion 22, and the alignment portion 24.

It can be appreciated that each of the first sheath 30 and/or the second sheath 32 may be translatable along the axis of the delivery catheter 12 to selectively cover or expose the respective region of the stent-valve 10, in response to actuation of a portion of a handle 34 of the delivery catheter 12. FIG. 1 illustrates that a proximal end region of the first sheath 32 may be coupled to a distal end of an outer shaft 35, whereby the proximal end of the outer shaft 35 may be attached to the handle 34.

Further, the first sheath 30 and the second sheath 32 may translate in opposite directions to one another. Additionally, the first sheath 30 and the second sheath 32 may translate in opposite directions to one another to expose the stent-valve 10 and/or to cover portions of the stent-valve 10. For example, the first (distal) sheath 30 may translate in a distal-to-proximal direction to expose the respective regions of the stent-valve 10 previously covered by the first sheath 30. Additionally, the first (distal) sheath 30 may translate in a proximal-to-distal direction to cover these regions during loading of the stent-valve 10 into the sheath 30. Similarly, the second (proximal) sheath 32 may translate in a distal-to-proximal direction to expose the respective regions of the stent-valve 10 previously covered by the second sheath 32. Additionally, the second (proximal) sheath 32 may translate in a proximal-to-distal direction to cover these regions during loading.

Further, the stent holder 28 may prevent, or at least reduce, a tendency of the stent-valve 10 to displace axially during translation of either the sheaths 30/32, and/or reduce any tendency of the stent-valve 10 to jump free of a respective sheath 30/32 when only a small portion of the stent-valve 10 is covered by the sheath 30. The stent holder 28 may be carried on a central shaft or tubular member 36, such as an inner tubular member for receiving a guide-wire.

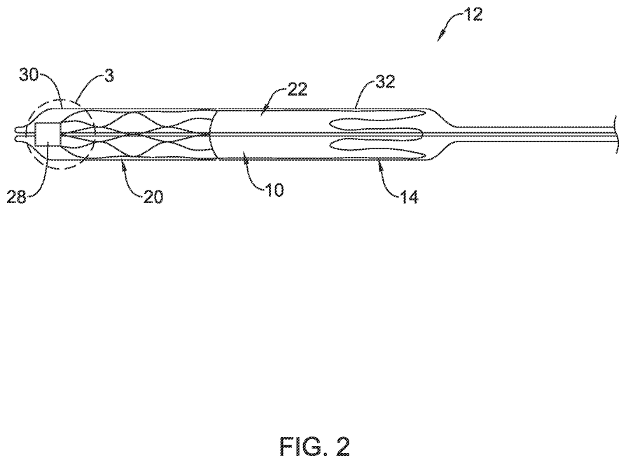

FIG. 2 illustrates the distal end region of the delivery catheter 12. Specifically, FIG. 2 illustrates the stent-valve 10 positioned within the first sheath 30 and the second sheath 32 of the delivery catheter 12. It can be appreciated that the delivery catheter 12 shown in FIG. 2 may be described as being in a pre-deployment configuration. In other words, FIG. 2 illustrates the delivery catheter 12 whereby the first sheath 30 is positioned over the anchoring portion 20 and/or a portion of the valve support portion 22 of the stent valve 10. Similarly, FIG. 2 illustrates the delivery catheter 12 whereby the second sheath 32 is positioned over the stent component 14 and/or a portion of the valve support portion 22. In this configuration, the delivery catheter 12 may be tracked over a guidewire to a target deployment site.

As will be described in greater detail below, loading the distal end region of the stent-valve 10 into the delivery catheter 12 may require the stent-valve 10 to be held stationary (in a compressed configuration) while the first sheath 30 is translated in a distal-to-proximal direction to a position at which the sheath 30 substantially covers the anchoring portion 20 and/or a portion of the valve support portion 22 of the stent valve 10. Additionally, loading the proximal end region of the stent-valve 10 into the delivery catheter 12 may require the stent-valve 10 to be held stationary (in a compressed configuration) while the first sheath 30 is translated in a proximal-to-distal direction to a position at which the sheath 30 substantially covers the stent component 14 and/or a portion of the valve support portion 22 of the stent-valve 10.

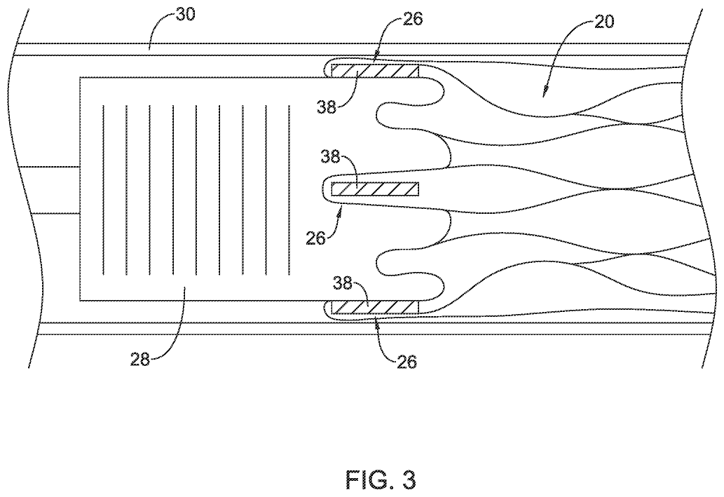

FIG. 3 illustrates a portion of the delivery catheter 12 illustrated in FIG. 2. Specifically, FIG. 3 illustrates the distal end region of the stent-valve 10 which is coupled to the stent holder 28. As shown in FIG. 3, the stent holder 28 may include a plurality of projections 38, each of which may extend radially away from the outer surface of the stent holder 28. In some instances, each projection 38 may resemble a “fin” extending away from the outer surface of the stent holder 28. However, this is not intended to be limiting. Rather, the projections 38 may include a variety of different shapes and/or geometric configurations designed to engage a portion of the distal end region of the stent-valve 10. Further, in some examples, the plurality of projections 38 may be spaced circumferentially around the outer surface of the stent holder 28.

Additionally, while FIG. 3 illustrates three projections 38 extending radially away from the outer surface of the stent holder 28, it is contemplated that the stent holder 28 may include more (or fewer) than three projections 38. For example, the stent holder 28 may include 1, 2, 3, 4, 5, 6 or more projections 38 designed to engage a portion of the distal end region of the stent-valve 10.

FIG. 3 further illustrates the attachment portions 26 of the stent-valve 10 engaged with the projections 38 of the stent holder 28. Specifically, FIG. 3 illustrates that an individual attachment portion 26 may extend distally away from a distal end of the anchoring portion 20 such that the attachment portion 26 may effectively loop around (e.g., hook, engage, etc.) an individual projection 38. It can be appreciated that the engagement of the attachment portions 26 with the projections 38 may be limit the ability of the stent-valve 10 to translate relative to the stent holder 28. It can be appreciated that, as discussed above, the stent holder 28 may be fixedly attached to the tubular member 36. Further, each of the first sheath 30 and the second sheath 32 may translate relative to the tubular member 36 and, consequently, the stent holder 28. Therefore, it can be appreciated that engagement of the attachment portions 26 of the stent-valve 10 with the projections 38 of the stent holder 28 may permit the first sheath 30 and the second sheath 32 to translate relative to the stent-valve 10.

As described above, loading the stent-valve 10 into the delivery catheter 12 (e.g., positioning the stent-valve 10 within the delivery catheter 12 as shown in FIG. 2) may require holding the stent-valve 10 in a radially-compressed configuration while the first sheath 30 and or the second sheath 32 is translated over the distal end region and the proximal end region the stent-valve 10, respectively. It can be further appreciated that, in some examples, it may be beneficial to utilize a medical device loading tool to hold the stent-valve 10 in a compressed configuration while the first sheath 30 and the second sheath 32 are translated over each of the distal end region and the proximal end region the stent-valve 10, respectively.

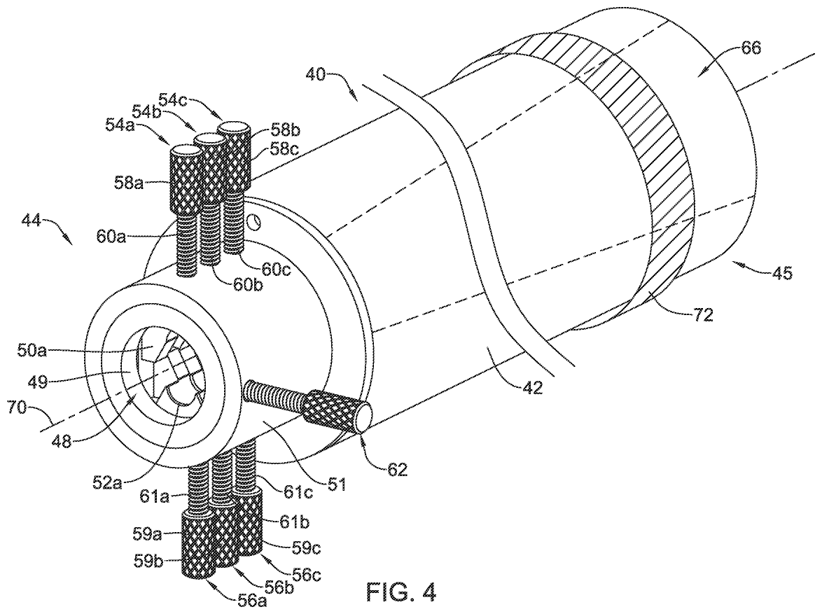

FIG. 4 illustrates an example medical device (e.g., stent-valve) loading tool 40 including a distal end region 44 and a proximal end region 45. Additionally, the loading tool 40 may include a cylindrical body 42 extending along the length of the loading tool 40 from the distal end region 44 of the loading tool 40 to the proximal end region 45 of the loading tool 40. The body 42 may further include a funnel-shaped lumen 66 extending therein. Further, the loading tool 40 may also include an advancement cap 72 which may be threaded onto the outer surface of the body 42. As will be described in greater detail below, the advancement cap 72 may include one or more projections which extend into the lumen 66 of the loading tool 40.

Additionally, FIG. 4 further illustrates that the distal end region 44 of the loading tool 40 may include a compression collar 51 which may extend distally away from the distal end of the body 42. As show in FIG. 4, the collar 51 may be include a generally cylindrical shape having an outer diameter that is less than the outer diameter of the body 42. While FIG. 4 illustrates that both the body 42 and the collar 51 may be cylindrically-shaped, this is not intended to be limiting. Rather, it is contemplated that each of the body 42 and the collar 51 may include different shapes. For example, the body 42 and/or the collar 51 may include square, ovular, triangular, rectangular, polygonal, or other similar shaped cross-sectional profiles.

FIG. 4 further illustrates that collar 51 may include a lumen 48 extending along the length of the collar 51. It can be appreciated that the lumen 48 may be aligned with the funnel-shaped lumen 66 of the body 42. In other words, while using the loading tool 40 to load a medical device (e.g., a stent-valve) into the delivery catheter, the delivery catheter 12 may be positioned such that it extends through the lumen 48 of the collar 51 and continues within (or just a portion of) the funnel-shaped lumen 66 of the body 42.

As will be described in greater detail herein, FIG. 4 illustrates that the compression collar 51 may further include a first plurality of compression pins 54a/54b/54c positioned on the collar 51. As illustrated in FIG. 4, the compression pins 54a/54b/54c may be aligned with one another along the longitudinal axis 70 of the loading tool 40. Further, each individual compression pin 54a/54b/54c may include a head portion and a threaded portion. For example, the individual compression pin 54a may include a head portion 58a and a threaded portion 60a, the individual compression pin 54b may include a head portion 58b and a threaded portion 60b and the individual compression pin 54c may include a head portion 58c and a threaded portion 60c.

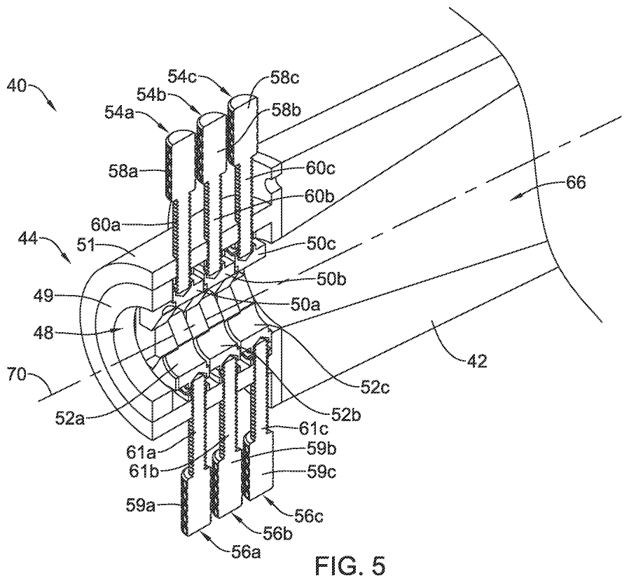

Further, as shown in FIG. 5, the threaded portions 60a/60b/60c of the compression pins 54a/54b/54c may extend through the wall of the collar 51, whereby each of the threaded portions 60a/60b/60c of the compression pins 54a/54b/54c may engage a compression element (e.g., V-block, iris, compression ring, etc.) positioned within the lumen 48 of the collar 51. For example, FIG. 4 illustrates a compression element 50a which may engage the threaded portion 60a of the compression pin 54a. It can be appreciated that the compression elements engaging the compression pins 54b and 54c are hidden from view in FIG. 4 (but are shown in FIG. 5).

Additionally, FIG. 4 illustrates that the compression collar 51 may further include a second plurality of compression pins 56a/56b/56c positioned along the collar 51. As illustrated in FIG. 4, the compression pins 56a/56b/56c may be aligned with one another along the longitudinal axis 70 of the loading tool 40. In some examples, the second plurality of compression pins 56a/56b/56c may be circumferentially spaced 180 degrees away from the first plurality of compression pins 54a/54b/54c.

Like the first plurality of compression pins 54a/54b/54c described above, each individual compression pin 56a/54b/54c may include a head portion and a threaded portion. For example, the individual compression pin 56a may include a head portion 59a and a threaded portion 61a, the individual compression pin 56b may include a head portion 59b and a threaded portion 61b and the individual compression pin 56c may include a head portion 59c and a threaded portion 61c.

Further, as is shown in FIG. 5, the threaded portions 61a/61b/61c of the compression pins 56a/56b/56c may extend through the wall of the collar 51, whereby each of the threaded portions 61a/61b/61c of the compression pins 56a/56b/56c may engage a compression element positioned within the lumen 48 of the collar 51. For example, FIG. 5 illustrates compression element 52a, which may engage the threaded portion 61a of the compression pin 56a. It can be appreciated that the compression elements engaging the compression pins 56b and 56c are hidden from view in FIG. 4 (but are shown in FIG. 5).

FIG. 4 further illustrates that the loading tool 40 may include a clamping pin 62. The clamping pin 62 may be utilized to hold a stop ring 49 in a fixed position. It can be appreciated from FIG. 5 that the stop ring 49 may function to prevent the compression elements 52a/52b/52c from shifting along the longitudinal axis 70. In other words, the stop ring 49 may function to prevent the compression elements 52a/52b/52c from falling out of the distal end of the collar 51.

FIG. 5 illustrates a cross-sectional view of the distal end region 44 of the loading tool 40. As described herein, FIG. 5 illustrates the threaded portions 60a/60b/60c of each of the compression pins 54a/54b/54c extending through the wall of the collar 51 to engage each of the compression elements 50a/50b/50c, respectively. Additionally, FIG. 5 illustrates the threaded portions 61a/61b/61c of each of the compression pins 56a/56b/56c extending through the wall of the collar 51 to engage each of the compression elements 52a/52b/52c, respectively.

It can be appreciated from FIG. 5 that each of the threaded portions 60a/60b/60c of each of the compression pins 54a/54b/54c and each of the threaded portions 61a/61b/61c of each of the compression pins 56a/56b/56c may engage with a threaded aperture in the collar 51 (e.g., each of the compression pins 54a/54b/54c/56a/56b/56c may be threaded through the wall of the collar 51). Accordingly, it can be appreciated that rotation of any given compression pin may translate that compression pin in a vertical direction (e.g., a direction which is perpendicular relative to the longitudinal axis 70 of the loading tool 40). Additionally, it can be further appreciated that because each compression pin is engaged with a corresponding compression element, the vertical translation of a particular compression pin (via rotation of that particular compression pin) may cause the vertical translation of the compression element with which the particular compression pin is engaged.

As an example, a user may rotate the head portion 58a of the compression pin 54a in a clockwise direction, whereby rotation of the head portion 58a in a clockwise direction may translate the threaded portion 60a in a vertical direction toward the longitudinal axis 70. Further, because the threaded portion 60a is engaged with the compression element 50a, the vertical translation of the threaded portion 60a may subsequently translate the compression element 50a a vertical direction toward the longitudinal axis 70. Additionally, it can be appreciated that rotating the compression pin in an opposite (e.g., counter-clockwise) direction, may reverse the translation of both the compression pin 54a and its corresponding compression element 50a, thereby translating the compression element 50a away from the longitudinal axis 70 of the loading tool 40. It can be appreciated that each of the compression pins 54b/54c/56a/56b/56c and their corresponding compression elements 50b/50c/52a/52b/52c may operate in in the same manner as that described above with respect to the compression pin 54a and its corresponding compression element 50a. Additionally, the “clockwise/counter-clockwise” rotation as described above is merely exemplary. It can be appreciated that the loading tool 40 may be designed such that “counter-clockwise” rotation of the pins 54a/54b/54c/56a/56b/56c may translate the pins 54a/54b/54c/56a/56b/56c toward the longitudinal axis 70 while “clockwise” rotation of the pins 54a/54b/54c/56a/56b/56c may translate the pins 54a/54b/54c/56a/56b/56c away from the longitudinal axis 70.

As discussed herein, rotation of one or more or the first plurality of compression pins 54a/54b/54c/56a/56b/56c may effectively translate the corresponding compression element 50a/50b/50c/52a/52b/52c with which the pin is engaged closer to or farther away from the longitudinal axis 70 of the loading tool. In some examples, the translation of a compression element 50a/50b/50c/52a/52b/52c away from the longitudinal axis 70 may be characterized as the “opening” of the compression element or while the translation of a compression element 50a/50b/50c/52a/52b/52c toward from the longitudinal axis 70 may be characterized as the “closing” of the compression element.

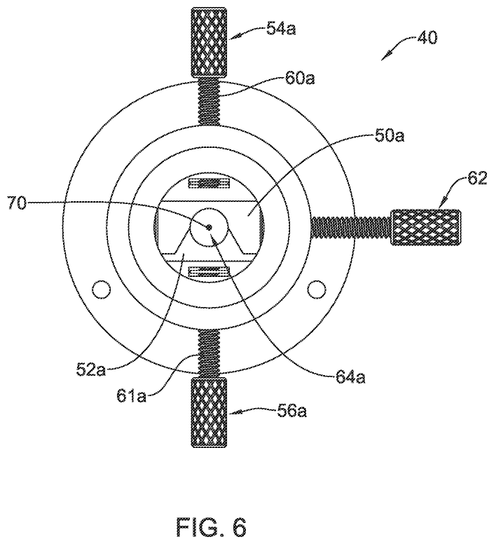

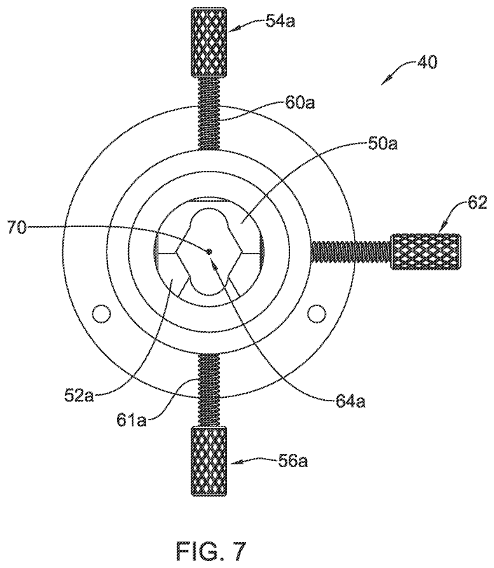

For example, FIGS. 6-7 illustrate the “opening” of the compression elements 50a/52a via the rotation of the compression pins 54a and 56a, respectively. Specifically,

FIG. 6 illustrates a front view of the loading tool shown in FIGS. 4-5, whereby each of the compression elements 50a/52a may characterized is being in a “compressed” or “closed” configuration. It can be appreciated that, in the compressed configuration, the compression pins 50a/52a may be rotated (e.g., in a clockwise direction) such that the compression elements 50a/52a have been translated to a position closer to the longitudinal axis 70 of the loading tool 40. Further, in the “compressed” configuration shown in FIG. 6, the compression elements 50a/52a may be spaced closer to the longitudinal axis 70 relative to the “open” configuration shown in FIG. 7. Further, it can be appreciated in the compressed configuration, the loading tool 40 may be include an aperture 64a defined between the compression element 50a and compression element 52a. Further, as will be described in greater detail below, in the compressed configuration, the aperture 64a may be specifically sized to radially compress the stent-valve 10 to a diameter designed to facilitate the loading of the stent-valve 10 onto the delivery catheter 12. Further yet, in the compressed configuration, the aperture 64a may be specifically sized to radially compress the stent-valve 10 to a diameter designed to permit the attachment portions 26 to engage the projections 38 of the stent holder 28.

As discussed herein, rotation of the compression pins 54a/56a may translate the compression members 50a/52a away from the longitudinal axis 70. For example, referring to FIG. 7, each of the compression pins 54a/56a may be rotated in a counter-clockwise direction, thereby translating each of the compression elements 50a/52a, respectively, away from the longitudinal axis 70 (e.g., shifting the compression elements 50a/52a from a compressed configuration shown in FIG. 6 to the open configuration shown in FIG. 7). Further, as will be described in greater detail below, in the open configuration, the aperture 64a has been enlarged relative to its size shown in FIG. 6, and may be sized to permit a portion of the delivery catheter 12 (e.g., the first sheath 30) to extend therein.

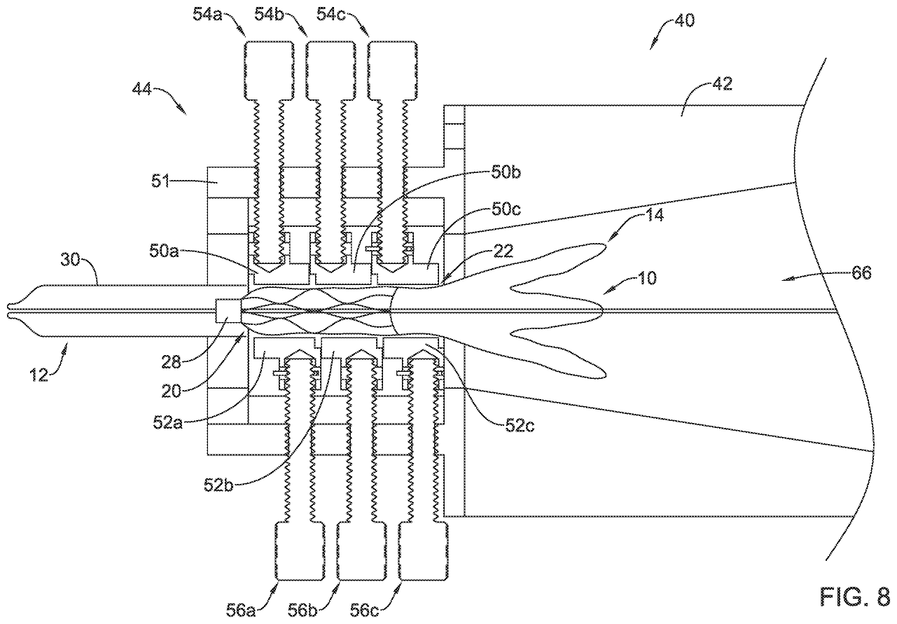

It can be appreciated from the discussion herein that each of the of the compression pins 54a/54b/54c/56a/56b/56c may effectively translate its corresponding compression element 50a/50b/50c/52a/52b/52c closer to or farther away from the longitudinal axis 70 of the loading tool. Accordingly, it can be further appreciated that while utilizing the loading tool 40 to load the stent-valve 10 onto the delivery catheter 12, it may be beneficial to open/close the compression elements 50a/50b/50c/52a/52b/52c sequentially. As will be described herein, loading a portion of the stent-valve 10 (e.g., the distal end region or proximal end region) into the delivery catheter 12 (e.g., the first sheath 30 or second sheath 32) may require that the stent-valve 10 be maintained in a radially compressed configuration while a portion of the delivery catheter 12 is incrementally positioned over the stent-valve 10. An example series of steps illustrating the incremental loading of the distal end region of the stent-valve 10 into the first sheath 30 of the delivery catheter is illustrated and described below with respect to FIG. 8-11.

FIG. 8 illustrates a first step to incrementally load the distal end region of the stent-valve 10 into the first sheath 30 of the delivery catheter 12. Specifically, FIG. 8 shows a cross-sectional view of the distal end region 44 loading tool 40, whereby portions of both the stent-valve 10 and delivery catheter 12 have been positioned within the collar 51 and the lumen 66 of the loading tool 40. It can be appreciated that prior to being positioned in the configuration shown in FIG. 8, the first sheath 30 and the second sheath 32 (not visible in FIG. 8) of the delivery catheter 12 may be spaced apart such that the stent-valve 14 may be positioned between the first sheath 30 and the second sheath 32.

It can be further appreciated that, prior to being positioned in the configuration shown in FIG. 8, the stent-valve 10 may have been advanced in a proximal-to-distal direction via actuation of the advancement cap 72. For example, the stent-valve 10 may initially be positioned within the full-shaped lumen 66 of the body 42. After being positioned in the funnel-shaped lumen 66, one or more projections coupled to the advancement cap 72 may extend into the lumen 66 and engage the proximal end region of the stent-valve 10. Additionally, actuation (e.g., rotation) of the advancement cap 72 may advance the cap 72 in a proximal-to-distal direction. Accordingly, actuation of the advancement cap 72 may advance the stent-valve 10 in a proximal-to-distal direction within the funnel-shaped lumen 66.

In some examples, the first step to incrementally load the distal end region of the stent-valve 10 into the first sheath 30 of the delivery catheter 12 may initially include advancing the stent-valve 10 distally such that the attachment portions 26 of the stent-valve 10 protrude just beyond the most distal compression element 50a. The stent holder 40 may then be positioned axially such that the attachment portions 26 of the stent-valve 10 are aligned and positioned directly above the projections 38 of the stent holder 28. After the attachment portions 26 of the stent-valve 10 are aligned and positioned directly above the projections 38 of the stent holder 28, the compression elements 50a/50b/50c/52a/52b/52c may then be closed to compress the distal portion of the stent-valve 10 to engage the attachment portions with the projections on the stent holder 28.

It can be further appreciated that the stent-valve 10 may continue to be radially compressed between the first plurality of compression elements 50a/50b/50c and the second plurality of compression elements 52a/52b/52c. In other words, the anchoring portion 20 and a portion of the valve support portion 22 may be “funneled” such that they are radially compressed between the first plurality of compression elements 50a/50b/50c and the second plurality of compression elements 52a/52b/52c. Further, the stent component 14 and a portion of the valve support portion 22 may remain within the lumen 66 of the body 42 of the loading tool 40.

Additionally, FIG. 8 illustrates that the proximal end of the first sheath 30 has been positioned within a portion of the lumen 48 of the collar 51, whereby the distal end of the first sheath 30 covers the stent holder 28 and abuts the distal end of the anchoring portion 20 of the stent-valve 10.

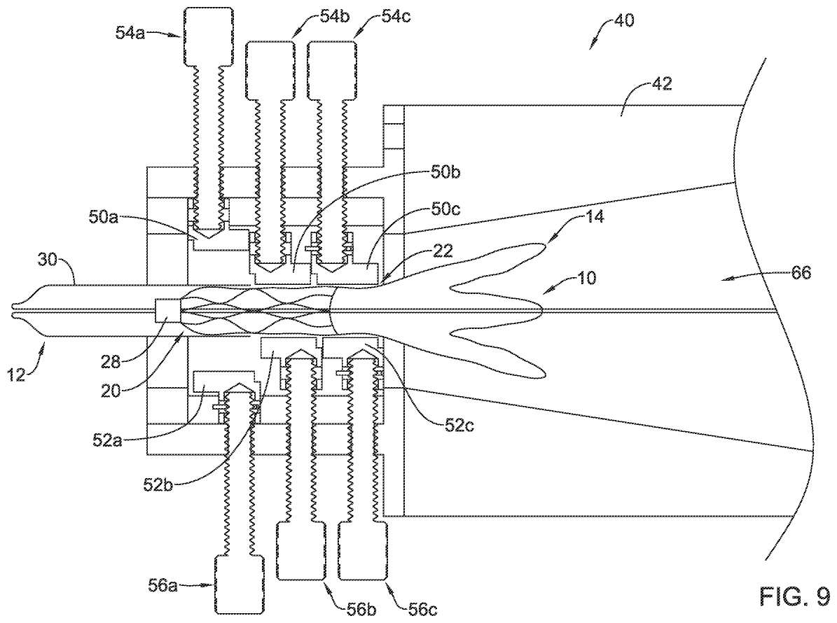

FIG. 9 illustrates a second step to incrementally load the distal end region of the stent-valve 10 into the first sheath 30 of the delivery catheter 12. Specifically, FIG. 9 shows the vertical translation of the compression element 50a and the compression element 52a away from the stent-valve 10 (e.g., the release of the compression element 50a and the compression element 52a from the stent-valve 10) followed by the advancement of the first sheath 30 over a portion of the anchoring portion 20 of the stent-valve 10. As described herein, translation of the compression element 50a and the compression element 52a away from the stent-valve 10 may be accomplished via the rotation of the compression pin 54a and the compression pin 56a, respectively.

Regarding the “sequential” process of advancing the first sheath 30 over the distal end region of the stent-valve 10, it can be appreciated that as the compression elements 50a/52a are being translated away from the stent-valve 10, the compression elements 50b/50c/52b/52c remain in place, thereby maintaining the stent-valve 10 in a radially compressed configuration. It can be appreciated that it may be beneficial to maintain the stent-valve 10 in a radially compressed configuration while a portion of the first sheath 30 is advanced over the portion of the anchoring portion 20 which had been radially compressed by the compression elements 50a/52a.

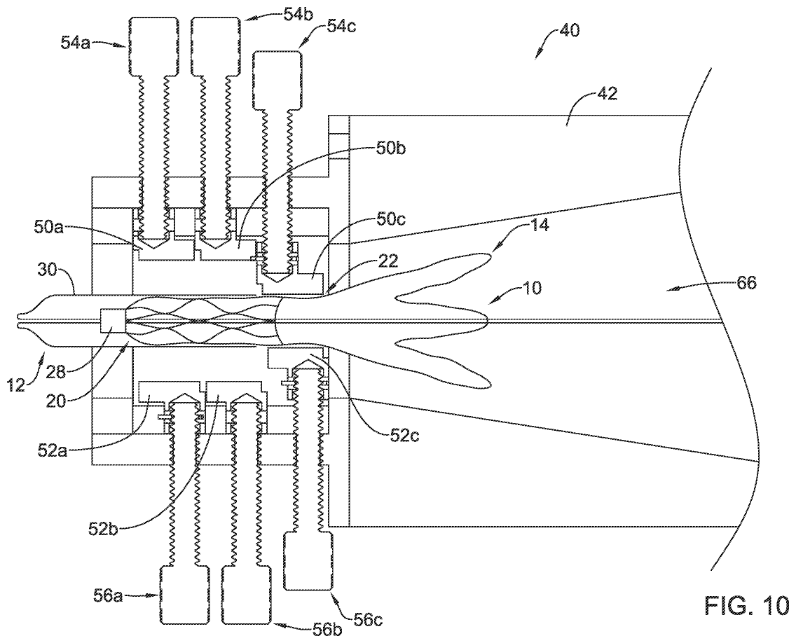

FIG. 10 illustrates a third step to incrementally load the distal end region of the stent-valve 10 into the first sheath 30 of the delivery catheter 12. Specifically, FIG. 10 shows the vertical translation of the compression element 50b and the compression element 52b away from the stent-valve 10 (e.g., the release of the compression element 50b and the compression element 52b from the stent-valve 10) followed by the further advancement of the first sheath 30 over a portion of the anchoring portion 20 of the stent-valve 10. As described herein, translation of the compression element 50b and the compression element 52b away from the stent-valve 10 may be accomplished via the rotation of the compression pin 54b and the compression pin 56b, respectively.

Additionally, it can be appreciated that as the compression elements 50b/52b are being translated away from the stent-valve 10, both the compression elements 50c/50c and the sheath 30 (which has been advanced over a portion of the anchoring portion 20 as described in the previous step) maintain the stent-valve 10 in a radially compressed configuration. It can be appreciated that it may be beneficial to continue to maintain the stent-valve 10 in a radially compressed configuration while the first sheath 30 is further advanced over the portion of the anchoring portion 20 which had been radially compressed by the compression elements 50a/50b/52a/52b.

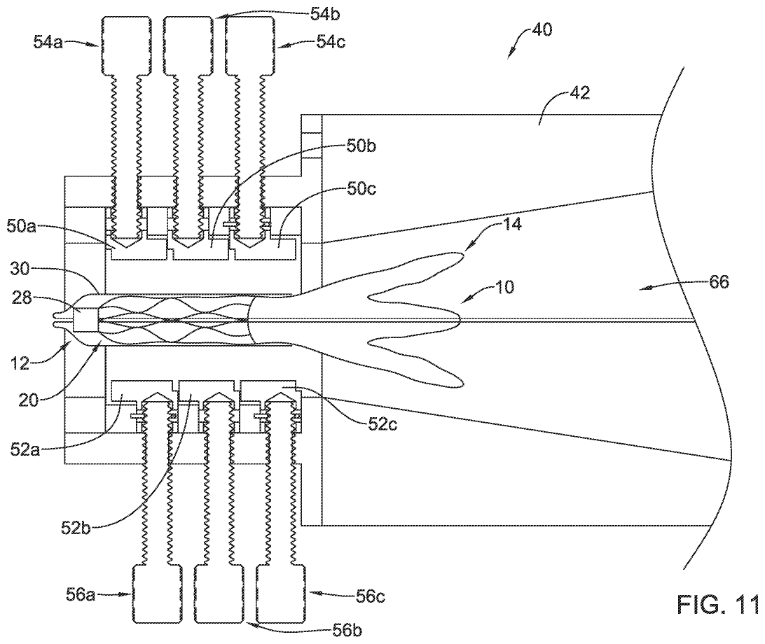

FIG. 11 illustrates a final step to incrementally load the distal end region of the stent-valve 10 into the first sheath 30 of the delivery catheter 12. Specifically, FIG. 11 shows the vertical translation of the compression element 50c and the compression element 52c away from the stent-valve 10 (e.g., the release of the compression element 50c and the compression element 52c from the stent-valve 10) followed by the further advancement of the first sheath 30 over a portion of the anchoring portion 20 and a portion of the valve support portion 22 of the stent-valve 10. As described herein, translation of the compression element 50c and the compression element 52c away from the stent-valve 10 may be accomplished via the rotation of the compression pin 54c and the compression pin 56c, respectively.

Additionally, it can be appreciated that as the compression elements 50c/52c are being translated away from the stent-valve 10, the sheath 30 (which has been further advanced over a portion of the anchoring portion 20 as described in the previous steps) maintains the stent-valve 10 in a radially compressed configuration. After completion of the final loading step described with respect to FIG. 11, the distal end region of the stent-valve 10 (including the anchoring portion 20 and a portion of the valve support portion 22) may be in loaded configuration such as that described with respect to FIG. 2.

Additionally, it can further be appreciated that the proximal end region of the stent-valve 10 may be loaded into the second sheath 32 of the delivery catheter 12 utilizing a sequential loading process similar to that described with respect to FIGS. 8-11. For example, the stent component 14 and/or a portion of the valve support portion 22 may be held in a radially compressed state (by the compression elements 50a/50b/50c/52a/52b/52c) while the compression elements 50a/50b/50c/52a/52b/52c are released sequentially as the second sheath 32 is incrementally advanced over the stent component 14 and/or a portion of the valve support portion 22 of the stent-valve 10.

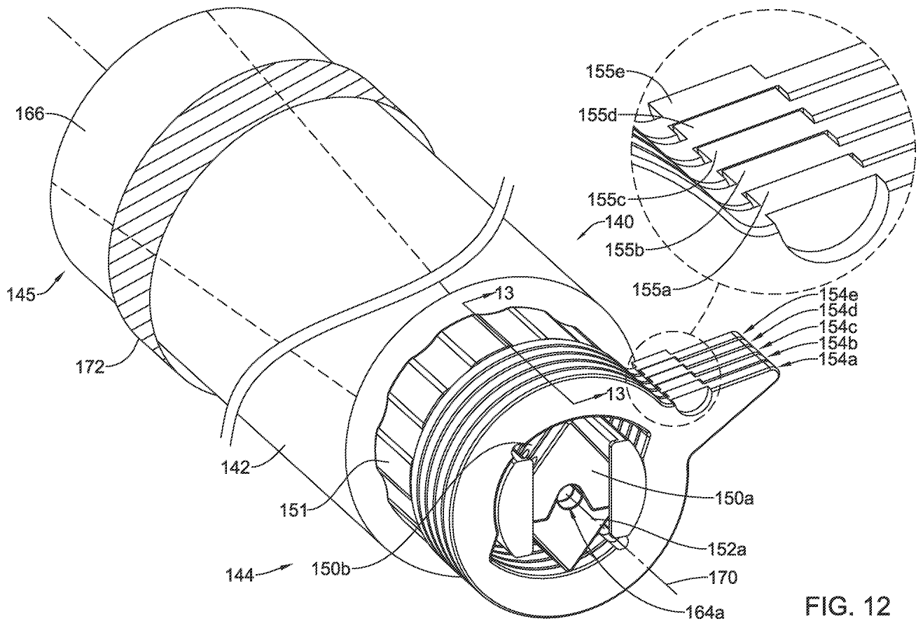

FIG. 12 illustrates another medical device (e.g., stent-valve) loading tool 140. The stent valve loading tool 140 may be similar in form and function as the loading tool 40 described herein. For example, the loading tool 140 may include a distal end region 144 and a proximal end region 145. Additionally, the loading tool 140 may include a cylindrical body 142 extending along the length of the loading tool 140 from the distal end region 144 of the loading tool 140 to the proximal end region 145 of the loading tool 140. The body 142 may further include a funnel-shaped lumen 166 extending therein. Further, the loading tool 140 may also include an advancement cap 172 which may be threaded onto the outer surface of the body 142. As will be described herein, the advancement cap 172 may include one or more projections which extend into the lumen 166 of the loading tool 140.

FIG. 12 further illustrates that the distal end region 144 of the loading tool 140 may include a collar 151 which may extend distally away from the distal end of the body 142. As show in FIG. 12, the collar 151 may be include a generally cylindrical shape having an outer diameter that is less than the outer diameter of the body 142. While FIG.

12 illustrates that both the body 142 and the collar 151 may be generally cylindrically-shaped, this is not intended to be limiting. Rather, it is contemplated that each of the body 142 and the collar 151 may include different shapes. For example, the body 142 and/or the collar 151 may include square, ovular, triangular, rectangular, polygonal, or other similar shaped cross-sectional profiles.

It can be appreciated that that collar 151 may include a lumen extending therein along the length of the collar 151. It can be further appreciated that the lumen of the collar may be aligned with the funnel-shaped lumen 166 of the body 142. In other words, while using the loading tool 140 to load a medical device (e.g., a stent-valve) into the delivery catheter 12, the delivery catheter 12 may be positioned such that it extends through the lumen of the collar 151 and continues within (or within a portion of) the funnel-shaped lumen 166 of the body 142.

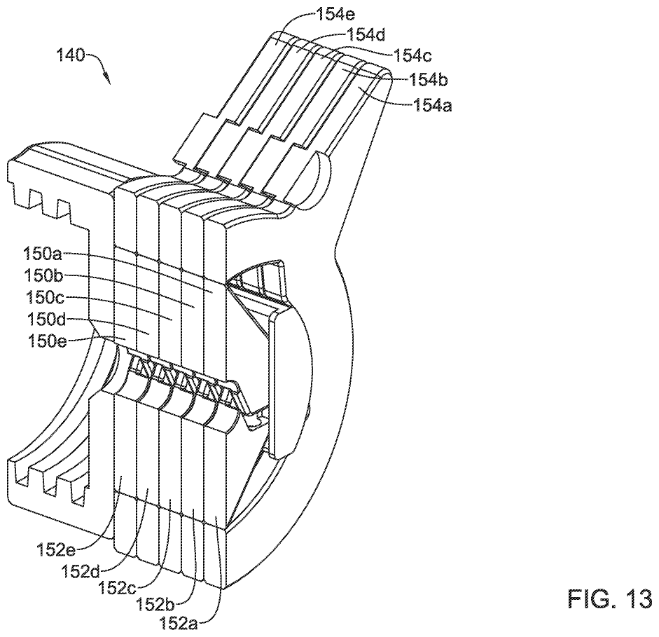

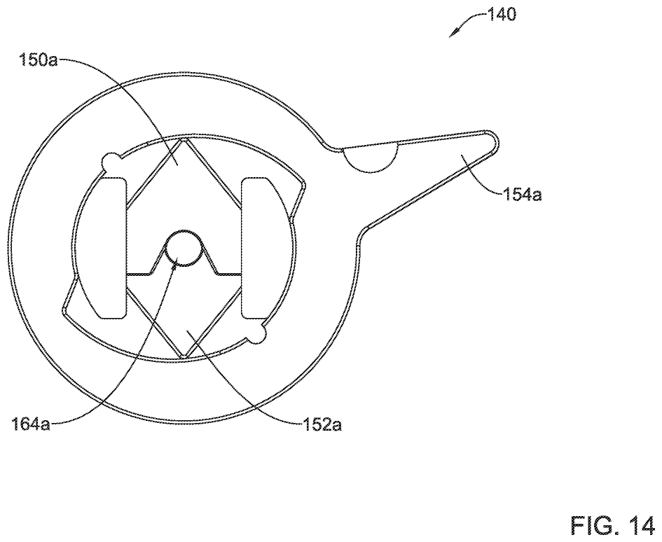

As will be described in greater detail herein, FIG. 12 further illustrates that the loading tool 140 may further include a plurality of cam members 154a/154b/154c/154d/154e coupled to the distal end of the collar 151. As shown in FIG. 12, each of the cam members 154a/154b/154c/154d/154e may be aligned with one another along the longitudinal axis 170 of the loading tool 140. Further, in some examples, each individual cam member 154a/154b/154c/154d/154e may be independently rotatable around the longitudinal axis 170 relative to the other cam members 154a/154b/154c/154d/154e. For example, as illustrated in FIGS. 13-14, the individual cam member 154a may be independently rotatable relative to any of the other cam members 154b/154c/154d/154e.

FIG. 12 further illustrates that each of the cam members 154a/154b/154c/154d/154e may be coupled to an upper compression member (e.g., an upper V-block) and a lower compression member. For example, FIG. 12 illustrates that the cam member 154a may be coupled to an upper compression member 150a and a lower compression member 152a. Additionally, FIG. 12 illustrates that a portion of the upper compression member 150b which may be coupled to the cam member 154b. FIG. 13 further illustrates each of the cam members 154a/154b/154c/154d/154e aligned with an upper compression member 150a/150b/150c/150d/150e and a lower compression member 152a/152b/152c/152d/152e.

It can be appreciated that actuation (e.g., rotation) of a specific cam member 154a/154b/154c/154d/154e may operate to translate both the upper compression member and the lower compression member to which the specific cam member is coupled. Like the compression members described above with respect to the loading tool 40, it can be appreciated that the rotation of a cam member 154a/154b/154c/154d/154e may translate its corresponding upper compression member and lower compression member toward or away from the longitudinal axis 170 of the loading tool 140. For example, FIG. 12 illustrates that the upper compression member 150a and the lower compression member 152a may define an aperture 164a. Further, FIG. 12 illustrates the cam member 154a (in addition to the remaining cam members 154b/154c/154d/154e) may be positioned such that the upper compression member 150a and the lower compression member 152a are in a “compressed” configuration, whereby the upper compression member 150a and the lower compression member 152a are closer to the longitudinal axis 170 relative to an “open” configuration in which they may be translated farther away from the longitudinal axis 170 (it is noted that FIG. 14 illustrates the cam 154a in an open position relative to the compressed position shown in FIGS. 12-13).

FIG. 12 further illustrates that each of the cam members 154a/154b/154c/154d/154e may include an extension portion 155a/155b/155c/155d/155e, whereby each of the extension portions 155a/155b/155c/155d extend longitudinally into a recess positioned in an adjacent cam member. For example, the cam member 154a includes an extension portion 155a which extends into a recess positioned in the cam member 154b. Similarly, the cam member 154b includes an extension portion 155b which extends into a recess positioned in the cam member 154c, the cam member 154c includes an extension portion 155c which extends into a recess positioned in the cam member 154d, and the cam member 154d includes an extension portion 155d which extends into a recess positioned in the cam member 154e.

It can be appreciated that the cam extension portion 155a/155b/155c/155d/155e function such that rotation of one of the cam members 154a/154b/154c/154d/154e in a clockwise direction will rotate all of the cam members 154a/154b/154c/154d/154e in a clockwise direction. In other words, when a user rotates a single cam member 154a/154b/154c/154d/154e in a clockwise direction, all the cam members 154a/154b/154c/154d/154e will close, thereby closing all of the upper compression members 150a/150b/150c/150d/150e and the lower compression members 152a/152b/152c/152d/152e. However, it can be further appreciated that each cam member 154a/154b/154c/154d/154e may be rotated counter-clockwise independently of all the other cam members 154a/154b/154c/154d/154e. It can be appreciated that permitting the cam members 154a/154b/154c/154d/154e to open independently of one another permits the incremental loading of the stent-valve into a portion of the delivery catheter.

It can be further appreciated that the cam members 154a/154b/154c/154d/154e, collectively, may operate to sequentially load a portion of the stent-valve 10 into a portion of the delivery catheter 12. For example, it can be appreciated that the distal end region of the stent-valve 10 (including the anchoring portion 20 and a portion of the valve support portion 22) may inserted into the central regions of the cam members 154a/154b/154c/154d/154e in a similar manner described above with respect to the loading tool 40. For example, the stent-valve 10 may be funneled within the funnel-shaped lumen 166 of the loading tool 140 via the proximal-to-distal advancement of the advancement cap 172. Accordingly, the funnel-shaped lumen 166 may radially compress the distal end region of the stent-valve 10 (including the anchoring portion 20 and a portion of the valve support portion 22) to aid in positioning the distal end region of the stent-valve 10 within the apertures of the cam members 154a/154b/154c/154d/154e.

As discussed herein, the radially compressed stent-valve 10 may be positioned with the individual apertures of each of the cam members 154a/154b/154c/154d/154e, whereby the cam members 154a/154b/154c/154d/154e may maintain the distal end region of the stent-valve 10 (including the anchoring portion 20 and a portion of the valve support portion 22) in a compressed configuration. Further, similar to that described above with respect to FIGS. 8-11, a portion of the delivery catheter 12 may extend within the cam members 154a/154b/154c/154d/154e, the collar 151 and the lumen 166 such that the distal end of the radially compressed stent-valve 10 may be aligned with the proximal end of the first sheath 30. Further yet, it can be appreciated that each individual cam member 154a/154b/154c/154d/154e may be incrementally opened (e.g., translated from the compressed configuration to the open configuration) to permit the sheath 30 to be advanced over the distal end region of the stent-valve 10, similarly to the loading methodology described above with respect to FIGS. 8-11.

FIG. 13 illustrates a cross-sectional view of the distal end region of the loading tool 140. FIG. 13 further illustrates each of the cam members 154a/154b/154c/154d/154e aligned with an upper compression member 150a/150b/150c/150d/150e and a lower compression member 152a/152b/152c/152d/152e. As discussed above, rotation of any cam member 154a/154b/154c/154d/154e in a clockwise direction will rotate all of the cam members 154a/154b/154c/154d/154e collectively, thereby opening the upper compression members 150a/150b/150c/150d/150e and the lower compression members 152a/152b/152c/152d/152e. Further, each individual cam member 154a/154b/154c/154d/154e may be incrementally opened (e.g., translated from the compressed configuration to the open configuration) when rotated in a counter-clockwise direction.

As discussed herein, FIG. 14 illustrates a front view of the loading tool 140 described above. As shown in FIG. 14, all the cam members 154a/154b/154c/154d/154e are in a compressed position (it is noted that the cam members 154b/154c/154d/154e are aligned with the leading cam member 154a and, therefore, are not visible in FIG. 14). FIG. 14 further illustrates the upper compression member 150a and the lower compression member 152a, which together define the aperture 164a.

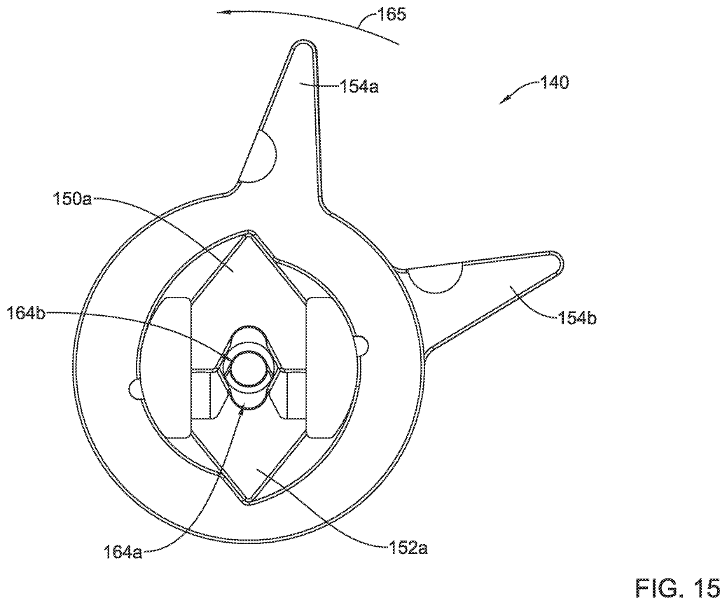

FIG. 15 illustrates the loading tool 140 in which the cam member 154a has been rotated from a compressed position to an open position. The rotation of the cam member 154a is depicted by the arrow 165 in FIG. 15. It can be appreciated from FIG. 15 that as the cam 154a is rotated from a compressed position to an open position, the cam 154b becomes visible in the illustration. It can be further appreciated from FIG. 15 that as the cam 154a is rotated from a compressed position to an open position, the aperture 164a defined by the upper compression member 150a and the lower compression member 152a enlarges. However, it is further noted that because none of the other cam members 154b/154c/154d/154e have been rotated, their respective apertures have not enlarged. For example, FIG. 15 illustrates the aperture of 164b which is defined by the upper compression member 150b and the lower compression member 152b of the cam 154b. It can be appreciated that the opening of a cam member (e.g., opening the cam member 154a) may correspond to a release of the compressed stent-valve 10, while the remaining cam members 154b/154c/154d/154e continue to maintain the stent-valve 10 in a radially compressed configured. As described above, as the cam members 154a/154b/154c/154d/154e are sequentially released, the first sheath 30 may be incrementally advanced over the distal end region of the stent-valve 10.

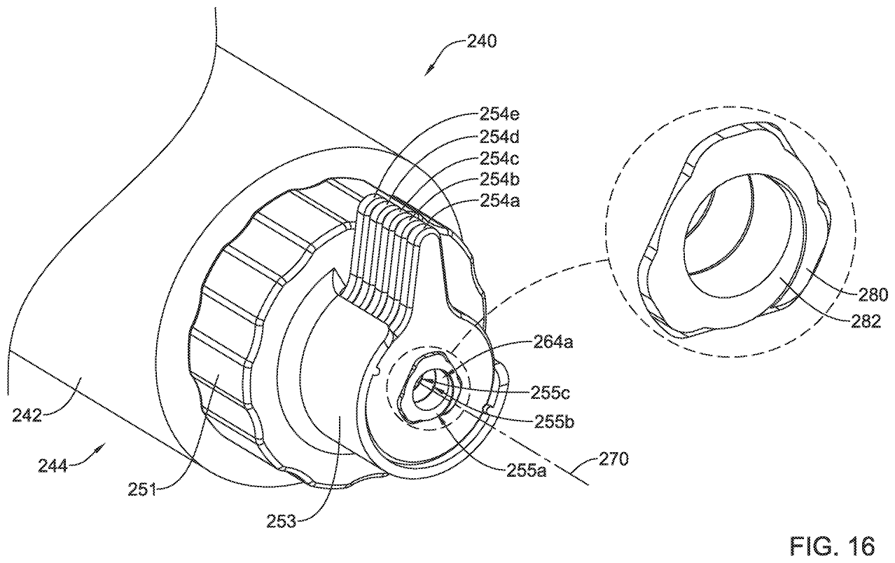

FIG. 16 illustrates the distal end region 244 of another example loading tool 240. The loading tool 240 may be similar in form and function to the loading tool 140 described above. For example, the loading tool 140 may include a cylindrical body 242 (similar to the body 142) including a funnel-shaped lumen extending therein. Further, the loading tool 240 may also include an advancement cap (similar to the advancement cap 172) which may be threaded onto the outer surface of the body 242. As will be described herein, the advancement cap may include one or more projections which extend into the funnel-shaped lumen of the loading tool 240.

FIG. 16 further illustrates that the distal end region 244 of the loading tool 240 may include a collar 251 which may extend distally away from the distal end of the body 242. As show in FIG. 16, the collar 251 may be include a generally cylindrical shape having an outer diameter that is less than the outer diameter of the body 242. While FIG. 6 illustrates that both the body 242 and the collar 251 may be generally cylindrically-shaped, this is not intended to be limiting. Rather, it is contemplated that each of the body 242 and the collar 251 may include different shapes. For example, the body 242 and/or the collar 251 may include square, ovular, triangular, rectangular, polygonal, or other similar shaped cross-sectional profiles.

Like the collar 151 of the loading tool 140, the collar 251 may include a lumen extending therein along its length. It can be further appreciated that the lumen of the collar may be aligned with the funnel-shaped lumen of the body 242. In other words, while using the loading tool 240 to load a medical device (e.g., a stent-valve) into the delivery catheter 12, the delivery catheter 12 may be positioned such that it extends through the lumen of the collar 251 and continues within (or within a portion of) the funnel-shaped lumen of the body 242. As will be described in greater detail herein, FIG. 16 further illustrates that the loading tool 240 may further include a plurality of cam members 254a/254b/254c/254d/254e coupled to the distal end of the collar 251. It can be further appreciated that the cam members 254a/254b/254c/254d/254e may function similarly to the cam members 154a/154b/154c/154d/154e. For example, each of the cam members 254a/254b/254c/254d/254e may rotate independently about the longitudinal axis 270 of the loading tool 240. However, in contrast to the loading tool 140 described herein, FIG. 16 illustrates that the loading tool 240 may include a plurality of compression rings positioned within the central region of each of the cam members 254a/254b/254c/254d/254e. For example, FIG. 16 illustrates a compression ring 255a which may be aligned with a central region of the cam member 254a, a compression ring 255b which may be aligned with a central region of the cam member 254b and a compression ring 255c which may be aligned with a central region of the cam member 254c. It can be appreciated that the loading tool 240 may also include compression rings which are aligned with the cam members 254d/254e, but are not visible in FIG. 16. It can be further appreciated that, in some examples, that the compression rings aligned with the cam members 254a/254b/254c/254d/254e may be disconnected from one another.

However, in other examples, it can be appreciated that the compression rings aligned with the cam members 254a/254b/254c/254d/254e may be connected to one another (e.g., connected via a spine member).

FIG. 16 further illustrates that the compression ring 255a (and the other compression aligned with the other cam members 254b/254c/254d/254e may be designed such that they are circumferentially discontinuous. For example, the detailed view of FIG. 16 illustrates that the compression ring 255a includes a first end portion 280 which overlaps a second end portion 282. It can be appreciated that because the first end portion 280 may not be connected to the second end portion 282, the first end portion 280 and the second end portion 282 may rotationally shift (e.g., slide) relative to one another. It can be further appreciated that as the first end portion 280 rotates relative to the second end portion 282, the size (e.g., diameter) of the aperture 264a defined by the compression ring 255a (including the first end portion 280 and the second end portion 282) may decrease and thereby apply a radially compressive force to a component (e.g., stent-valve) extending therein.

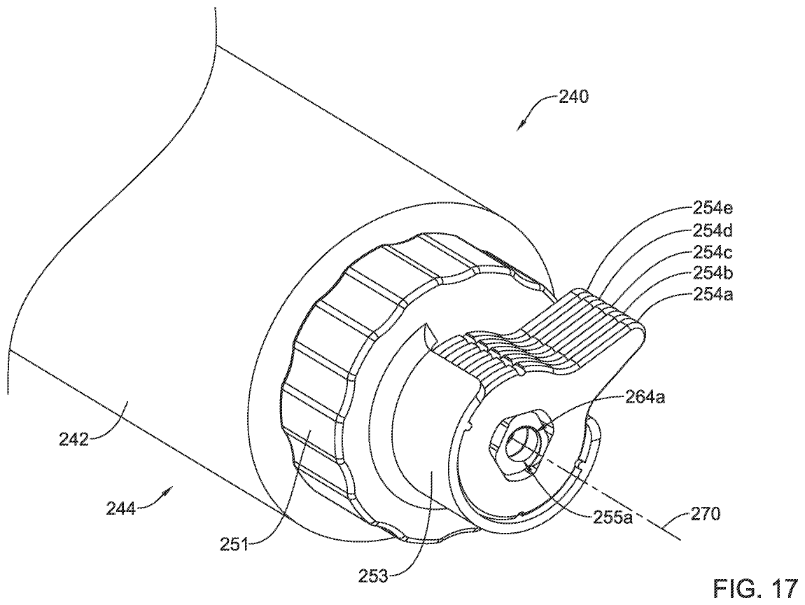

FIG. 17 illustrates the loading tool 240 after each of the cam members 254a/254b/254c/254d/254e have been rotated around the longitudinal axis 270 from an open configuration (shown in FIG. 16) to the compressed configuration. As described herein, it can be appreciated that rotation of each of the cam members 254a/254b/254c/254d/254e may decrease the aperture of the compression ring coupled to each of the cam members 254a/254b/254c/254d/254e. For example, FIG. 17 illustrates that the size of the aperture 264a of the compression ring 255a has been decreased. As discussed above, as the size of the aperture 264a decreases, it may apply a radially compressive force to a component (e.g., stent-valve) extending therein.

It can be further appreciated from FIG. 16 and FIG. 17 that as the cam 254a is rotated from a compressed position to an open position, the aperture 264a defined by the upper compression ring 255a enlarges. However, it is further noted that if the other cam members 254b/254c/254d/254e remain in a compressed position, their respective apertures will not be enlarged. Therefore, like the loading tool 140 described herein, it can be appreciated that the opening of a particular cam member (e.g., opening the cam member 254a) may correspond to a release of a compressed stent-valve 10, while the remaining cam members 254b/254c/254d/254e continue to maintain the stent-valve 10 in a radially compressed configured. As described above with respect to the loading methodology of the loading tools 40/140, as the cam members 254a/254b/254c/254d/254e are sequentially released, the first sheath 30 may be incrementally advanced over the distal end region of the stent-valve 10.

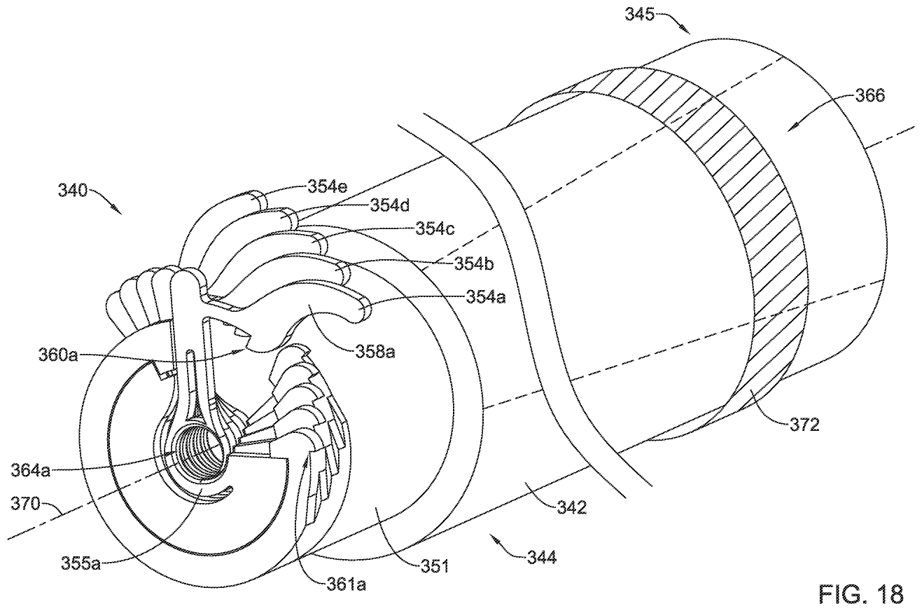

FIG. 18 illustrates another medical device (e.g., stent-valve) loading tool 340. The stent valve loading tool 340 may be similar in form and function as other loading tools described herein. For example, the loading tool 340 may include a distal end region 344 and a proximal end region 345. Additionally, the loading tool 340 may include a cylindrical body 342 extending along the length of the loading tool 340 from the distal end region 344 of the loading tool 340 to the proximal end region 345 of the loading tool 340. The body 342 may further include a funnel-shaped lumen 366 extending therein. Further, the loading tool 340 may also include an advancement cap 372 which may be threaded onto the outer surface of the body 342. As will be described herein, the advancement cap 372 may include one or more projections which extend into the lumen 366 of the loading tool 340.

FIG. 18 further illustrates that the distal end region 344 of the loading tool 340 may include a collar 351 which may extend distally away from the distal end of the body 342. As show in FIG. 18, the collar 351 may be include a generally cylindrical shape having an outer diameter that is less than the outer diameter of the body 342. While FIG. 18 illustrates that both the body 342 and the collar 351 may be generally cylindrically-shaped, this is not intended to be limiting. Rather, it is contemplated that each of the body 342 and the collar 351 may include different shapes. For example, the body 342 and/or the collar 351 may include square, ovular, triangular, rectangular, polygonal, or other similar shaped cross-sectional profiles.

It can be appreciated that that collar 351 may include a lumen extending therein along the length of the collar 351. It can be further appreciated that the lumen of the collar 351 may be aligned with the funnel-shaped lumen 366 of the body 342. In other words, while using the loading tool 340 to load a medical device (e.g., a stent-valve) into the delivery catheter 12, the delivery catheter 12 may be positioned such that it extends through the lumen of the collar 351 and continues within (or within a portion of) the funnel-shaped lumen 366 of the body 342.

As will be described in greater detail herein, FIG. 18 further illustrates that the loading tool 340 may further include a plurality of compression levers 354a/354b/354c/354d/354e coupled to the distal end of the collar 351. As shown in FIG. 13, each of the compression levers 354a/354b/354c/354d/354e may be aligned with one another along the longitudinal axis 370 of the loading tool 340. Further, in some examples, a portion of each individual compression lever 354a/354b/354c/354d/354e may rotate about the longitudinal axis 370.

FIG. 18 further illustrates that each individual compression lever 354a/354b/354c/354d/354e may include an actuation arm, a first locking projection, a second locking projection and an integrated compression portion. For example, FIG. 18 illustrates the compression lever 354a including an actuation arm 358a, a first locking projection 360a, a second locking projection 361a and a compression portion 364a.

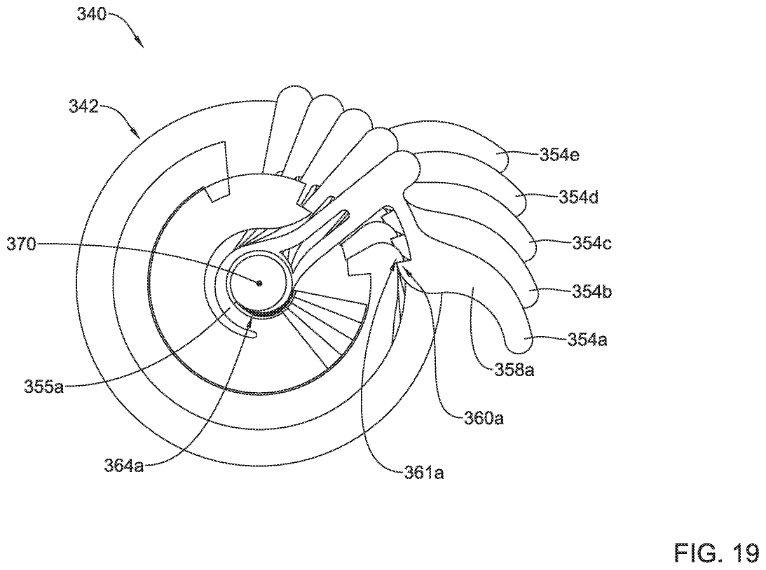

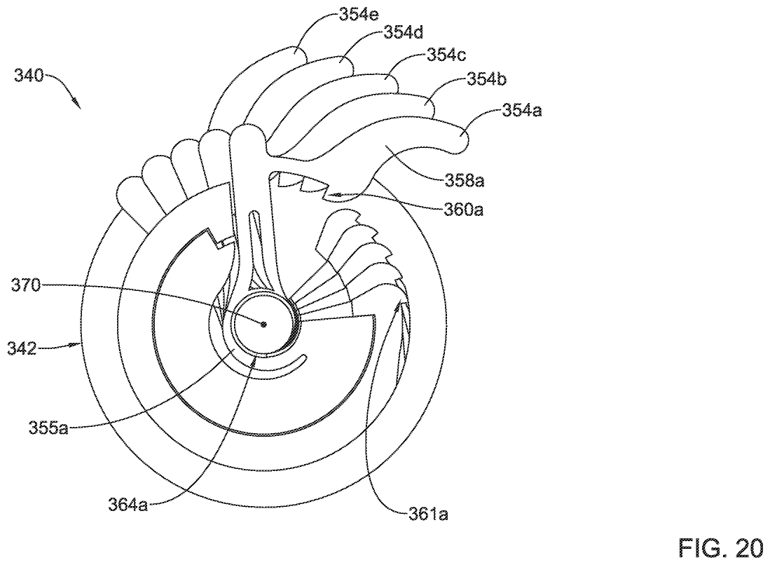

Further, FIG. 18 illustrates each of the compression levers 354a/354b/354c/354d/354e in an open configuration. It can be appreciated that the actuation arm of each compression lever 354a/354b/354c/354d/354e may be rotated around the longitudinal axis 370 to shift the compression portions of each compression lever 354a/354b/354c/354d/354e between the open configuration and a compressed configuration (it is noted that FIG. 19 illustrates the compression levers 354a/354b/354c/354d/354e in a compressed configuration relative to the open configuration as illustrated in FIGS. 18 and 20.)

Referring to the compression lever 354a shown in FIG. 18 as an example, it can be appreciated from that the actuation arm 358a may be rotated about the longitudinal axis 370. Further, FIG. 18 further illustrates that the compression portion 364a may be designed such that it is circumferentially discontinuous (similar to the compression ring 264a described above). Therefore, it can be appreciated that as the actuation arm 358a rotates around the longitudinal axis 370, the size (e.g., diameter) of the aperture 364a defined by the compression portion 355a may decrease and thereby apply a radially compressive force to a component (e.g., stent-valve) extending therein.

As discussed herein, FIG. 19 illustrates a front view of the loading tool 340 described above. As shown in FIG. 19, all the compression levers 354a/354b/354c/354d/354e are in a compressed configuration. It can be further appreciated from FIG. 19 that, when in a compressed configuration, the first projection and the second projection of each compression lever may engage with one another to releasably “lock” the compression lever in the compressed configuration. For example, FIG. 19 illustrates the first projection 360a engaged with the second projection 361a to releasably lock the compression lever 354a in a compressed position. It can be appreciated from FIG. 19 that each of the compression levers 354b/354c/354d/354e may also releasably lock in the compressed position.

FIG. 20 illustrates the loading tool 340 in which the first projection and the second projection have been disengaged to permit each of the compression levers 354a/354b/354c/354d/354e to shift from the compressed configuration (shown in FIG. 19) to an open configuration (shown in FIG. 20). It can be appreciated by comparing FIG. 19 and FIG. 20, that as the actuation arm 358a is rotated from a compressed configuration (shown in FIG. 19) to an open configuration (shown in FIG. 20), the aperture 364a defined by the compression portion 355a enlarges. However, it is further noted that if the other compression levers 354b/354c/354d/354e remain in a compressed position, their respective apertures will not be enlarged. Therefore, like the loading tool 340 described herein, it can be appreciated that the opening of a particular compression lever (e.g., opening the compression lever 354a) may correspond to a release of a compressed stent-valve 10, while the remaining compression levers 354b/354c/354d/354e continue to maintain the stent-valve 10 in a radially compressed configured. As described above with respect to the loading methodology of other loading tools described herein, as the compression levers 354b/354c/354d/354e are sequentially released, the first sheath 30 may be incrementally advanced over the distal end region of the stent-valve 10. It is further noted that the compression levers 354b/354c/354d/354e may maintain the stent-valve 10 in a radially compressed configuration as a user incrementally disengages “releases” each actuation arm (via disengagement of the first projection and the second projection of each compression lever) to shift each compression lever from the compressed configuration to the open configuration.

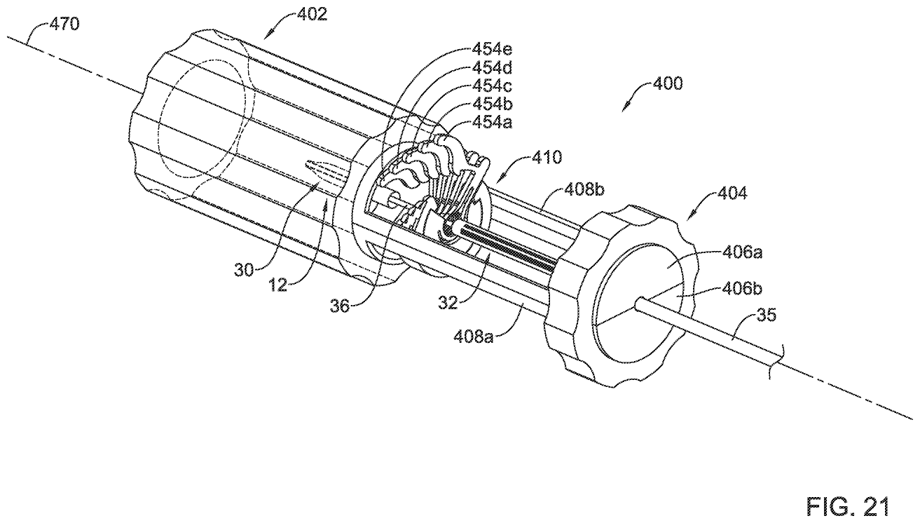

FIG. 21 illustrates an example proximal sheath loading tool 400. The proximal sheath loading tool 400 may be utilized to advance the proximal sheath 32 (described with respect to FIGS. 1-2 herein) over the proximal portions of the example stent-valve 10 (described with respect to FIGS. 1-2 herein). For example, the proximal sheath loading tool 400 may be utilized to advance the proximal sheath 32 over a portion of the anchoring portion 20, the valve support portion 22, and the alignment portion 24 of the stent-valve 10.

FIG. 21 illustrates that the proximal sheath loading tool 400 may include a distal end region which includes a first rotation member 402. The first rotation member 402 may include an inner lumen which permits a portion of the stent-valve delivery system 12 to extend therein. For example, FIG. 21 illustrates the distal sheath 30 and a portion of the inner tubular member 36 extending within a lumen of the first rotation member 402. As will be described in greater detail below, it can further be appreciated that the first rotation member 402 may freely rotate around a portion of the delivery system 12.

FIG. 21 further illustrates that the first rotation member 402 may be coupled to a first rail member 408a and a second rail member 408b. Additionally, the distal ends of each of the first rail member 408a and the second rail member 408b may be coupled to the first rotation member 402. Further yet, it can be appreciated that rotation of the first rotation member 402 may shift the first rail member 408a and the second rail member 408b along the longitudinal axis 470 of the proximal sheath loading tool 400. For example, rotation of the first rotation member 402 may translate (e.g., pull) each of the first rail member 408a and the second rail member 408b toward the first rotation member 402.

FIG. 21 further illustrates that the proximal sheath loading tool 400 may include a proximal end region which includes a second rotation member 404. The second rotation member 404 may include a first collet component 406a and a second collet component 406b. As shown in FIG. 21, each of the first collet component 406a and the second collet component 406b may be nested within central region of the first rotation member 404. It can be appreciated that rotation of the second rotation member 404 may actuate the first collet component 406a relative to the second collet component 406b. For example, rotation of the second rotation member 404 in a first direction may move the first collet component 406a away from second collet component 406b while rotation of the second rotation member 404 in a second direction (e.g., opposite the first direction) may move the first collet component 406a toward the second collet component 406b. As described herein, it can be appreciated that because the first rotation member 402 is coupled to the second rotation member 404 (via the first rail member 408a and the second rail member 408b), rotation of the first rotation member 402 may translate the second rotation member 404 along the longitudinal axis 470. For example, rotation of the first rotation member 402 may translate the second rotation member 404 along the longitudinal axis 470 toward the first rotation member 402 (e.g., rotation of the first rotation member 402 may pull the second rotation member 404 toward the first rotation member 402).

Additionally, FIG. 21 illustrates that the proximal loading tool 400 may include a multi-clamp component 410 positioned adjacent the proximal end of the first rotation member 402. The multi-clamp component 410 may be further positioned between the first rail member 408a and the second rail member 408b. The multi-clamp component 410 may include one or more components which are similar to the loading tool 340 described herein. Specifically, the multi-clamp component 410 may include a plurality of compression levers 454a/454b/454c/454d/454e which are are designed to shift between a compressed configuration and an open configuration (similar to the compression levers 354a/354b/354c/354d/354e described herein). Further, like the compression levers 354a/354b/354c/354d/354e described herein, the compression levers 454a/454b/454c/454d/454e may be designed to be incrementally actuated (e.g., opened individually in sequence).

FIG. 21 illustrates a portion of the stent-valve delivery system 12 positioned within the proximal sheath loading tool 400 (for clarity, the stent-valve 10 is not shown positioned within the delivery system 12). For example, FIG. 21 illustrates the distal sheath 30 positioned within the lumen of the first rotation member 402, while the proximal sheath 32 is positioned proximal to the multi-clamp component 410. It can be appreciated that the configuration of the delivery system 12 relative to the loading tool 400 shown in FIG. 21 may resemble a configuration in which the distal portion of the stent-valve 10 (not shown in FIG. 21, but it can be appreciated that a portion of the stent-valve 10 may be loaded into the delivery system 12) has already been loaded into the distal sheath 30 (e.g., as shown in FIG. 2 and described with respect to FIGS. 8-11), while the proximal portion (e.g., the anchoring portion 20, the valve support portion 22, and the alignment portion 24) of the stent-valve 10 remain to be loaded into the proximal sheath 32.

It can be appreciated that to load the proximal portion (e.g., the anchoring portion 20, the valve support portion 22, and the alignment portion 24) of the stent-valve 10 into the proximal sheath 32, the multi-clamp component 410 may initially hold the proximal portion (e.g., the anchoring portion 20, the valve support portion 22, and the alignment portion 24) of the stent-valve 10 in a compressed configuration. An example next step may include rotation of the second rotation member 404 such that the first collet component 406a moves toward the second collet 406b, thereby securing the outer shaft 35 between the first collet component 406a and the second collet component 406b.

Further, and as described above, the distal end of the outer shaft 35 may be coupled to the proximal end of the proximal sheath 32. Therefore, translation of the first rotation member 402 may also translate the proximal sheath 32 along the longitudinal axis (e.g., rotation of the first rotation member 402 may pull the proximal sheath toward the first rotation member 402 and the multi-clamp component 410).

Therefore, an example next step to load the proximal portion (e.g., the anchoring portion 20, the valve support portion 22, and the alignment portion 24) of the stent-valve 10 into the proximal sheath 32 may include rotating the first rotation member 402 such that the proximal sheath 32 is translated to a position in which the distal end of the proximal sheath 32 is adjacent to the multi-clamp component 410.

An example next step to load the proximal portion (e.g., the anchoring portion 20, the valve support portion 22, and the alignment portion 24) of the stent-valve 10 into the proximal sheath 32 may include opening the compression lever 454a. It can be appreciated that opening the compression lever 454a may expose a portion of the alignment portion 24 of the stent-valve 10 (e.g., it may expose the portion of stent-valve 10 which was being compressed by the compression lever 454a). However, the exposed portion of the stent-valve 10 may still be held in a compressed configuration by the remaining compression levers 454b/454c/454d/454e.

Accordingly, it can be appreciated that an example next step in loading the proximal portion of the stent-valve 10 into the proximal sheath 32 may include rotating the first rotation member 402 to further pull a portion of the proximal sheath 32 into the compression lever 454a and over the exposed portion of the alignment portion 24 (e.g., pull the proximal sheath 32 over the portion of the stent-valve 10 which was being compressed by the compression lever 454a).

It can be further appreciated that the step-wise, incremental opening of the remaining compression levers 454b/454c/454d/454e, followed by the incremental rotation of the first rotation member 402 to pull the proximal sheath 32 over the respective exposed portions of the stent-valve 10, may incrementally load the entire proximal portion (e.g., the anchoring portion 20, the valve support portion 22, and the alignment portion 24) of the stent-valve 10 into the proximal sheath 32.

It is contemplated that the proximal sheath loading tool 400 illustrated in FIG. 21 may utilize other embodiments described herein to incrementally compress and release the proximal portion of the stent-valve 10. For example, the proximal sheath loading tool 400 may utilize the compression pins and compression elements described with respect to FIGS. 4-11 and/or the cam members described with respect to FIGS. 12-17 to incrementally compress and release the proximal portion of the stent-valve 10.

The materials that can be used for the various components of the medical devices 40/140/240/340/400 and the various other medical devices disclosed herein may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP).

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A loading device for a stent-valve, comprising:
an elongated body having a proximal end region, a distal end region and a lumen extending therein;
a collar coupled to the distal end region of the body, the collar including first end region, a second end region and a lumen extending therein;
a first compression assembly coupled to the collar, wherein the first compression assembly is configured to shift between a first position a second position; and
a second compression assembly coupled to the collar, wherein the second compression assembly is configured to shift between a third position and a fourth position.

2. The loading device of claim 1, wherein the first compression assembly is longitudinally offset from the second compression assembly.

3. The loading device of claim 1, wherein the first compression assembly and the second compression assembly are configured to maintain the stent-valve in a radially compressed state when in the first position.

4. The loading device of claim 3, wherein shifting first compression assembly, the second compression assembly, or both the first compression assembly and the second compression assembly from the first position to the second position includes releasing the stent-valve from the radially compressed state.

5. The loading device of claim 4, wherein the first compression assembly is configured to release the stent-valve from the radially compressed state while the second compression assembly maintains the stent-valve in radially compressed state.

6. The loading device of claim 5, wherein the second compression assembly is configured to release the stent-valve from the radially compressed state after the first compression assembly releases the stent-valve from the radially compressed state.

7. The loading device of claim 1, wherein the first compression assembly includes a first compression member coupled to a first actuation member.

8. The loading device of claim 3, wherein actuation of the first actuation member shifts the first compression member between the first position and the second position.

9. The loading device of claim 7, further comprising a second compression member coupled to a second actuation member, and wherein both the first actuation member and the second actuation member include a threaded pin.

10. The loading device of claim 7, wherein the first actuation member includes a cam.

11. The loading device of claim 7, wherein the first actuation member includes compression lever.

12. The loading device of claim 11, wherein the compression lever includes a first projection and a second projection, and wherein the first projection is configured to releasably engage with the second projection.

13. The loading device of claim 7, wherein the first compression member includes a compression ring, and wherein the circumference of the compression ring is circumferentially discontinuous.

14. The loading device of claim 13, wherein the compression ring includes an aperture having a first diameter when the first compression assembly is in the first position, and wherein the aperture has a second diameter when the first compression assembly is in the second position, and wherein the first diameter is less than the second diameter.

15. A loading device for a stent-valve, comprising:
an elongated body having a proximal end region, a distal end region and a conical lumen extending therein;
a collar coupled to the distal end region of the body, the collar including first end region, a second end region and a lumen extending therein;
a first compression assembly coupled to the collar, a second compression assembly coupled to the collar, wherein the first compression assembly is longitudinally offset from the second compression assembly;
wherein the first compression assembly and the second compression assembly are configured to maintain the stent-valve in a radially compressed state while in a first position;
wherein the first compression assembly is configured to release the stent-valve from the radially compressed state while the second compression assembly maintains the stent-valve in the radially compressed state.

16. The loading device of claim 15, wherein the first compression assembly includes a first compression member coupled to a first actuation member.

17. The loading device of claim 16, wherein the first actuation member includes a cam.

18. The loading device of claim 16, wherein the first actuation member includes compression lever.

19. The loading device of claim 16, wherein the first compression member includes a compression ring, and wherein the circumference of the compression ring is circumferentially discontinuous.

20. A method of loading a stent-valve on to a stent-valve delivery device, the method comprising:
positioning a first sheath of the stent-valve delivery device adjacent to a stent-valve loading device, the stent-valve loading device including:
an elongated body;
a collar coupled to the distal end region of the body;
a first compression assembly coupled to the collar, the first compression assembly including a first compression region; and
a second compression assembly coupled to the collar, the second compression assembly including a second compression region; and
compressing the stent-valve within the first compression region and the second compression region;
releasing the stent from the first compression region;
advancing the first sheath of the stent-valve delivery device over a first portion of the stent-valve;
releasing the stent from the second compression region; and
advancing the first sheath of the stent-valve delivery device over a second portion of the stent-valve.
